(19) United States
(12) Patent Application Publication
(10) Pub. No.: US 2004/0092784 A1
(43) Pub. Date: May 13, 2004

(54) Catalytic conversion of alkanes to alkenes

(76) Inventor: Olivier Legendre, Herblay (FR)

Correspondence Address: BURNS DOANE SWECKER & MATHIS L L P, POST OFFICE BOX 1404, ALEXANDRIA, VA, 22313-1404, US

(21) Appl. No.: 10/239,358

(22) PCT Filed: Mar. 23, 2001
(86) PCT No.: PCT/EP01/03954

(30) Foreign Application Priority Data
Mar. 24, 2000 (EP) 00106398.1

(51) Int. Cl. C07C 5/333

(52) U.S. Cl. 585/660; 585/661

(57) ABSTRACT

A process for the catalytic conversion of an alkane to an alkene comprising reacting a C₂ to C₅ alkane and a sulfur-containing compound at a temperature of from 300° C. to 650° C. in the presence of a catalyst having a surface area greater than 100 square meters per gram, to afford the corresponding C₂ to C₅ alkene and hydrogen sulfide.

The present invention relates to a catalytic process for the conversion of an alkane to the corresponding alkene and in particular to a process for the cataytic conversion of an alkane and sulphur to the corresponding alkene and hydrogen sulphide.

The catalytic dehydrogenation of alkanes to alkenes is a well known reaction but the commercial operation of this reaction has suffered problems. In particular under the operating conditions used in this reaction there is often the unwanted side reaction of cracking of high alkane which results in the production ethene and methane. Furthermore, low conversion of the alkane leads to the necessary additional separation step to isolate the desired alkene product.

U.S. Pat. No. 3,801,661 discloses a process for the dehydrogenation of non-aromatic C₃ to C₅ alkanes to the corresponding alkene. The hydrocarbon is contacted with a metal sulphide catalyst. The reaction requires the presence of hydrogen sulphide and steam. These components are not co-reactants but are essential to maintain the stability of the catalyst under the fairly severe operating conditions, in particular the reaction temperature which is 700° C. The conversion rate in this process is 70% and thus an additional separation step is necessary to isolate the alkane product.

U.S. Pat. No. 3,456,026 discloses the sulphur dehydrogenation of organic compounds. In particular this patent discloses a process for the dehydrogenation of alkanes to alkenes. The process of this patent may be carried out at temperature in excess of 800° F. although in reality the operating temperature is in excess of 1000° F. The catalyst used in the dehydrogenation has a surface area of between 0.01 and 100 square metres per gram of catalyst. The patent states that the catalyst must have low surface area to avoid cracking and tar formation.

U.S. Pat. No. 3,787,517 discloses the oxidative dehydrogenation of an alkane by the vapour phase catalytic reaction with carbonyl sulphide. The preferred catalyst is an iron based catalyst. This patent is silent as regards the surface area of the catalyst.

We have found that high conversion of the alkane and high selectivity to the corresponding alkene can be achieved when the alkane is reacted with a sulphur containing compound in the presence of a catalyst under less severe reaction conditions and in particular with the specific combination of low temperature and a catalyst having a high surface area.

Accordingly, the present invention provides a process for the catalytic reaction of a C₂ to C₅ alkane and a sulphur containing compound to produce the corresponding alkene and hydrogen sulphide wherein the reaction mixture is contacted with a catalyst at a temperature of from 300 to 650° C. wherein the catalyst has a surface area greater than 100 square metres per gram.

The process of the present invention provides the advantage over the prior art in that a higher proportion of the converted alkanes is the desired alkenes Thus, the downstream separation steps are simplified. Furthermore, operation of the process at a relatively low temperature reduces the amount of unwanted side reactions.

The process of the present invention is directed to the reaction between a C₂ to C₅ alkane and a sulphur containing compound to produce the corresponding alkene and hydrogen sulphide. By the “corresponding alkene” is meant the unsaturated product having the same number of carbon atoms as the feed hydrocarbon. A particularly preferred alkane feed for use in the present invention is propane and thus the reaction between propane and a sulphur containing compound to provide propene and hydrogen sulphide is particularly preferred.

The process of the present invention may be carried out in the gas phase or in the liquid phase. It is preferred to carry out the process in the gas phase.

The sulphur containing compound used in the process of the present invention is a compound which is able to react with the alkane to yield hydrogen sulphide. Suitable sulphur containing compounds include sulphur oxides, namely sulphur dioxide and sulphur trioxide, H₂SO₃, H₂SO₄, ammonium sulphite, ammonium sulphate, elemental sulphur or a mixture thereof The sulphur containing compound may be present in the reaction mixture in the liquid or gaseous form. Preferably, the sulphur containing compound is present as gaseous sulphur.

The molar ratio of sulphur to alkane is suitably from 0.1 to 10 moles to of sulphur to 1 mole of alkane, preferably from 0.2 to 5 moles of sulphur to 1 mole of alkane, especially from 0.25 to 0.5 moles of sulphur to 1 mole of alkane.

Inert diluents such as nitrogen, noble gases e.g. helium and argon, carbon monoxide, hydrogen sulphide and carbon disulphide or a mixture thereof may be included in the reaction mixture. The inert diluent may be present in a total concentration of from 0 to 95%, preferably from 0 to 75% of the mixture.

A preferred reaction mixture is 10% propane, 5% gaseous sulphur and 85% nitrogen.

The catalyst used in the process of the present invention may be selected from the known dehydrogenation catalysts. Suitable dehydrogenation catalysts include metallic sulphides, in particular where the metal is a metal from Groups V B, VI B, VII B, VIII B and Group I B of the Periodic Table. Examples of sulphide catalysts include tungsten sulphide, nickel sulphide, molybdenum sulphide, copper sulphide and cobalt sulphide. The metal sulphide catalyst may comprise a mixture of two or more metals. Suitable catalysts falling into this category include tungsten/nickel, molybdenum/nickel and molybdenum/cobalt sulphides. The preferred metal sulphide catalyst is a cobalt/molybdenum sulphide catalyst. The metal sulphide catalyst may be introduced into the reactor in the sulphide form or alternatively, may be introduced in another form which is capable of being converted to the sulphide form, for example the oxide form may be used and treated with a mixture of hydrogen and hydrogen sulphide prior to use.

Metal oxide catalysts may also be used in the process of the present invention and in particular oxides of Group VI B and of aluminium. Preferably, the metal oxide catalyst is aluminium oxide and chromium oxide. The metal oxide catalyst may comprise two or more metals and in particular a mixture of molybdenum and chromium is preferred.

The metal sulphide or metal oxide catalyst may be supported on a support. Suitable supports include alumina, titania, zirconia, silica, aluminosilicates or a mixture thereof. Preferably the catalyst support is alumina.

A further class of materials capable of catalysing the process of the present invention is carbon-based materials such as activated carbon. These materials optionally may be promoted with a suitable active material such as metal sulphides.

A further class of compounds that have been founf to be suitable for use in the present invention are alumino siklicates, in particular zeolites and especially ZSM-5, promotes with a Group I or Group II metal such as lithium or magnesium.

The catalyst used in the process of the present invention has a surface area greater than 100 square metres per gram. The actual surface area may vary according to the support or carrier used with the catalyst. For example where the catalyst is a metal sulphide or metal oxide, preferably, the catalyst has a surface area of greater than 100 and less than 400 m²/g, especially between 200 and 300 m²/g. Where the catalyst is carbon-based, the surface area may be greater that 100 m²/g and less than 600 m²/g

After a long periods of time in use, the catalyst may need to be regenerated. The regeneration may be carried out by passing gaseous sulphur over the catalyst at the reaction temperature for a suitable period of time. Typically, the sulphur is contacted with the catalyst for 10 to 15 hours.

A particular advantage of the present invention is that the process can be operated under mild reaction conditions. The process is operated at a temperature of from 300 to 650° C., more preferably from 450 to 580° C. Good conversion per pass and selectivity of product are obtained when the process is operated at 550° C.

The process may be operated at any suitable pressure, for example below atmospheric, above atmospheric or at atmospheric pressure. Suitably, the process may be operated at a pressure of from 0.05 to 50 bar, preferably from 0.1 to 20 bar.

The space velocity is suitably from 50 to 5000 h⁻¹, preferably from 500 to 1500 h⁻¹. It will of course be apparent to the person skilled in the art that the space velocity will vary according to the temperature and pressure.

The process may be operated in any suitable reactor capable of handling the heat transfer to the catalyst bed. Suitable reactors include multi-tubular reactor, a standard reactor equipped with an internal heating coil or a simple adiabatic reactor.

The process may be operated batchwise, semi-continuously or continuously. It is preferred to operate the process as a semi-continuous or continuous process.

It is well known by the person skilled in the art that as a result of consecutive reactions, the higher the conversion of alkane the lower will be the selectivity to alkene products. Thus, in the present process it is preferred to separate the alkene product from the unreacted alkane and recycle the alkane on a continuous basis. Suitable, the recycle ratio of unreacted alkane to reacted alkane if s from 1 to 10, preferable from 3 to 5 recycle the unreacted alkane

The products of the present process are predominantly the alkane and hydrogen sulphide. Overall conversion of the alkane is typically from 90 to 95% with a recycle ratio of from 3 to 5. The conversion per pass is typically from 15 to 35%. Selectivity to the alkene is typically greater than 50%, preferably greater than 90%, especially greater that 95%. A small amount of by-products are present in the product stream such as hydrogen, methane, ethane, ethene and carbon disulphide. These by-products are present in small quantities, typically from 100 to 50000 ppm volume and may be separated by any simple method, for example distillation.

The present invention will now be illustrated with reference to the following examples:

### EXAMPLE 1

### Conversion of Propane and Sulphur to Propene and Hydrogen Sulphide using Alumina Catalyst

A glass reactor was loaded with 8.3 ml of alumina particles, having a surface area of 190 m²/g. A gaseous mixture of sulphur and propane, molar ratio of 0.56 to 1, was fed into the reactor at a space velocity of 1200 h⁻¹. The gas flow rate was 10 litres per hour. Helium was also introduced into the reactant stream as a diluent to obtain a concentration of propane of 10.4%

The reactor was heated to 550° C. and the process operated under a pressure of 1.05 bar for approximately 5 hours on stream.

The gaseous product stream was analysed on a continuous basis by gas chromatography. The analyses indicated 36 to 45% conversion of propane and from 100 to 90% conversion of sulphur. The propene selectivity varied from 44 to 53%.

### EXAMPLE 2

### Conversion of Propane and Sulphur to Propene and Hydrogen Sulphide using Chromium Oxide Catalyst

A glass reactor was loaded with 6 ml of chromium oxide, having a surface area of 250 m²/g. A gaseous mixture of sulphur and propane, molar ratio of 0.90 to 1, was fed into the reactor at a space velocity of 520 h⁻¹. The gas flow rate was 3.2 litres per hour. Helium was also introduced into the reactant stream as a diluent to obtain a concentration of propane of 10.7%

The reactor was heated to 450° C. and the process operated under a pressure of 1.04 bar for approximately 7 hours on stream.

The gaseous product stream was analysed on a continuous basis by gas chromatography. The analyses indicated 20 to 7% conversion of propane and complete conversion of sulphur. The propene selectivity varied from 90 to 50%.

### EXAMPLE 3

### Conversion of Propane and Sulphur to Propene and Hydrogen Sulphide using Nickel/Tungsten Sulphide Catalyst on Alumina

A nickel/tungsten oxide catalyst having a surface area of 180 m²/g, was treated with a mixture of hydrogen and hydrogen sulphide at a temperature not exceeding 350° C. for 6 hours to provide the nickel/tungsten sulphide form.

A glass reactor was loaded with 13.8 ml of nickel/tungsten sulphide on alumina. A gaseous mixture of sulphur and propane, molar ratio of 0.19 to 1, was fed into the reactor at a space velocity of 1170 h⁻¹. The gas flow rate was 16.2 litres per hour. Helium was introduced into the reactant stream as a diluent to obtain a concentration of propane of 13.4%

The reactor was heated to 555° C. and the process operated under a pressure of 1.04 bar for approximately 12 hours on stream.

The gaseous product stream was analysed on a continuous basis by gas chromatography. The analyses indicated 46 to 14% conversion of propane and between 80 and 93% conversion of sulphur. The propene selectivity varied from 50 to 74%.

### EXAMPLE 4

### Conversion of Propane and Sulphur to Propene and Hydrogen Sulphide using Vanadium Pentoxide Catalyst

An alumina carrier having a surface area of 190 m²/g was promoted with vanadium pentoxide by wet impregnation of vanadyl oxalate, followed by calcination at 500° C.

A glass reactor was loaded with 8.3 ml of the vanadium pentoxide. A gaseous mixture of sulphur and propane, molar ratio of 0.57 to 1, was fed into the reactor at a space velocity of 900 h⁻¹. The gas flow rate was 7.5 litres per hour. Helium was introduced into the reactant stream as a diluent to obtain a concentration of propane of 10.2%

The reactor was heated to 550° C. and the process operated under a pressure of 1.03 bar for approximately 5 hours on stream.

The gaseous product stream was analysed on a continuous basis by gas chromatography. The analyses indicated 82 to 36% conversion of propane and from 100 to 90% conversion of sulphur. The propene selectivity varied from 38 to 60%.

### COMPARATIVE EXAMPLE 1

### Conversion of Propane and Sulphur to Propene and Hydrogen Sulphide using Alumina Catalyst with Low Surface Area

A glass reactor was loaded with 8.3 ml of alumina particles, having a surface area of 72 m²/g. A gaseous mixture of sulphur and propane, molar ratio of 0.56 to 1, was fed into the reactor at a space velocity of 1200 h⁻¹. The gas flow rate was 10 litres per hour. Helium was also introduced into the reactant stream as a diluent to obtain a concentration of propane of 10.3%

The reactor was heated to 550° C. and the process operated under a pressure of 1.05 bar for approximately 3 hours on stream.

The gaseous product stream was analysed on a continuous basis by gas chromatography. The analyses indicated 1.3 to 0% conversion of propane and negligeable conversion of sulphur. The propene selectivity could not be accurately measured.

1. A process for the catalytic reaction of a C₂ to C₅ alkane and a sulphur containing compound to produce the corresponding alkene and hydrogen sulphide wherein the reaction mixture is contacted with a catalyst at a temperature of from 300 to 650° C. wherein the catalyst has a surface area greater than 100 square metres per gram.

2. A process as claimed in claim 1 carried out at a temperature of from 450 to 580° C.

3. A process as claimed in claim 1 or claim 2 in which the catalyst has a surface area of from 100 to 600 square metres per gram.

4. A process as claimed in any one of the preceding claims in which the alkane and the sulphur containing compound is present in a mole ratio of from 0.1 to 10 moles of sulphur to 1 mole of alkane.

5. A process as claimed in claim 4 in which the alkane and the sulphur containing compound is present in a mole ratio of from 0.25 to 0.5 moles of sulphur to 1 mole of alkane.

6. A process as claimed in any one of the preceding claims in which the alkane is propane.

7. A process as claimed in any one of the preceding claims in which the sulphur containing compound is elemental sulphur.

8. A process as claimed in any one of the preceding claims in which the catalyst is a metallic sulphide wherein the metal is selected from Groups V B, VI B, VII B, VIII B and Group I B of the Periodic Table

9. A process as claimed in claim 7 in which the catalyst is a metallic sulphide is selected from tungsten sulphide, copper sulphide, nickel sulphide, molybdenum sulphide cobalt sulphide, tungsten/nickel sulphide, molybdenum/nickel sulphide and molybdenum/cobalt sulphide or a mixture thereof.

10. A process as claimed in claim 9 in which the catalyst is tungsten/nickel sulphide.

11. A process as claimed in any one of claims 1 to 7 in which the catalyst is a metal oxide wherein the metal is selected from Group VI B of the Periodic Table or alumina.

12. A process as claimed in claim 11 in which the catalyst is chromium oxide or alumina.

13. A process as claimed in any one of the preceding claims in which the catalyst comprises a support selected from alumina, titania, zirconia, silica, aluminosilicates or a mixture thereof.

14. A process as claimed in any one of the preceding claims carried out under a pressure of from 0.05 to 50 bar and a space velocity of from 50 to 5000 h⁻.

15. A process as claimed in any one of the preceding claims wherein any unreacted alkane is recycled to the reactor.
